(19) United States
(12) Patent Application Publication
(10) Pub. No.: US 2004/0158291 A1
(43) Pub. Date: Aug. 12, 2004

(54) Implantable heart monitors having electrolytic capacitors with hydrogen-getting materials

(76) Inventors: Jeannette C. Polkinghorne, St. Anthony, MN (US); James M. Poplett, Plymouth, MN (US); Gregory J. Sherwood, North Oaks, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/361,132

(22) Filed: Feb. 7, 2003

(51) Int. Cl. A61N 1/39

(52) U.S. Cl. 607/5

(57) ABSTRACT

Implantable heart monitors, such as defibrillators and cardioverters, detect abnormal heart rhythms and automatically apply corrective electrical shocks to the hearts of patients. A critical component in these devices are the capacitors that produce the electrical shocks. One problem with some of these capacitors is that during operation they generate internal gases, which over time accumulate and exert pressure on their cases, often forcing the cases to swell or bulge and potentially compromising capacitor and monitor performance. Accordingly, the inventors devised novel capacitors that include titanium and/or other hydrogen-getting materials and structures, for preventing the development of excessive pressures within capacitor cases.

### TECHNICAL FIELD

The present invention concerns implantable heart monitors, such as defibrillators and cardioverters, particularly structures and methods for capacitors in such devices.

### BACKGROUND

Since the early 1980s, thousands of patients prone to irregular and sometimes life-threatening heart rhythms have had miniature heart monitors, particularly defibrillators and cardioverters, implanted in their bodies, specifically in the upper chest area above their hearts. These devices detect onset of abnormal heart rhythms and automatically apply corrective electrical therapy, specifically one or more bursts of electric current, to hearts. When the bursts of electric current are properly sized and timed, they restore normal heart function without human intervention, sparing patients considerable discomfort and often saving their lives.

The typical defibrillator or cardioverter includes a set of electrical leads, which extend from a sealed housing into the walls of a heart after implantation. Within the housing are a battery for supplying power, monitoring circuitry for detecting abnormal heart rhythms, and at least one capacitor for delivering bursts of electric current through the leads to the heart.

The capacitor is often times an aluminum electrolytic capacitor, which takes a flat or cylindrical form. The flat form of this type capacitor generally includes a stack of flat capacitor elements or modules, each comprising two or more aluminum foils and an electrolyte-soaked separator between them. The stack of flat modules, often D-shaped, are housed in a sealed aluminum case of similar shape. The cylindrical form includes one long capacitor module that is rolled up and housed in a round tubular, or cylindrical, aluminum case.

One problem with both the flat and cylindrical forms of these capacitors is that during normal operation their capacitor modules electro-chemically generate gases, such as hydrogen, that are trapped inside the sealed cases. Over the life of some of these capacitors, the trapped gases accumulate and exert considerable pressure on the cases, often forcing them to swell and permanently distort. This swelling is problematic not only because of the cramped spacing within implantable heart monitors, but also because it causes portions of some foils to separate from adjacent separators and to be starved of electrolyte. This starvation increases equivalent series resistances (ESR) and reduces capacitance, or energy-storage capacity, of the capacitors.

To address this problem, some capacitor manufacturers have sought to make their sealed cases with thicker walls to resist swelling. However, the inventors have recognized that this solution is of limited value because it often increases the size and weight of capacitors and/or reduces the space available for components, such as aluminum foil, which contribute to the total capacitance, or energy-storage density, of the capacitors. Additionally, some capacitor manufacturers have introduced organic nitro-compounds to the electrolyte of the capacitor to reduce production of hydrogen gas. However, these compounds have not proven to successfully reduce hydrogen gas build-up in all cases.

Accordingly, the inventors identified an unmet need for better ways of avoiding or reducing capacitor swelling, particularly for capacitors in implantable heart monitors.

### SUMMARY

To address this and other needs, the inventors devised novel structures and related capacitors and devices that include hydrogen- or other gas-getting materials and thus prevent the development of excessive pressures within their cases. One exemplary capacitor includes at least aluminum and titanium. Another exemplary capacitor includes the titanium in the form of a titanium and titanium-oxide coating on an aluminum cathode. In this embodiment, the titanium absorbs or adsorbs hydrogen gas, and the titanium oxide, which has a much higher dielectric constant than the aluminum oxide present in conventional aluminum electrolytic capacitors, increases capacitance.

Other aspects of the invention include an implantable heart monitor, such as pacemaker, defibrillator, cardioverter, or defibrillator-cardioverter, which comprises one or more of the novel capacitors or other related structures.

### BRIEF DESCRIPTION OF THE DRAWINGS

FIG. 1 is a cross-sectional view of an exemplary structure embodying the present invention.

FIG. 2 is a perspective view of an exemplary flat aluminum electrolytic capacitor 100 including a generic pressure-relief mechanism 120, embodying the present invention.

FIG. 3 is a perspective view of an exemplary cylindrical electrolytic capacitor 200 including a generic pressure-relief mechanism 220 embodying the present invention.

FIG. 4 is a block diagram of an exemplary implantable heart monitor 400 embodying the present invention.

### DETAILED DESCRIPTION OF PREFERRED EMBODIMENTS

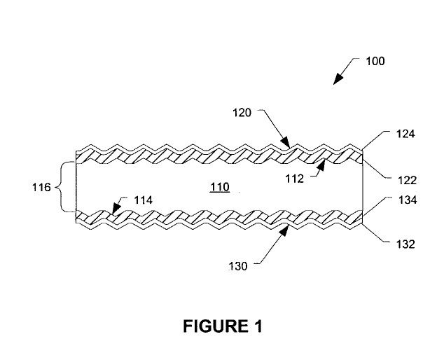

The following detailed description, which incorporates FIGS. 1-4 and the appended claims, describes and illustrates one or more specific embodiments of the invention. These embodiments, offered not to limit, but to exemplify and teach, are shown and described in sufficient detail to enable those skilled in the art to implement or practice the invention. Thus, where appropriate to avoid obscuring the invention, the description may omit certain information known to those of skill in the art.

FIG. 1 shows an exemplary structure 100 incorporating teachings of the present invention. Structure 100 includes an aluminum substrate 110 and coat structures 120 and 130.

Aluminum substrate 110 has opposing major surfaces 112 and 114, which define a nominal thickness 116. In the exemplary embodiment, aluminum substrate 110 consists essentially of a commercially available high-purity aluminum, and nominal thickness 116 lies in the range of 5-150 micrometers (im.) (Other embodiments use other thicknesses, aluminum concentrations, and possibly even other base metals.) Also in the exemplary embodiment, surfaces 112 and 114 are roughened by chemical etching or other suitable procedure. In some embodiments, the roughened surfaces have an effective surface area 2-5 times that of the “unroughened” surface, and still other embodiments have an effective surface area 200-300 times that of the unroughened surface. Affixed respectively to surfaces 112 and 114 are coat structures 120 and 130.

Coat structure 120 includes a non-aluminum hydrogen-absorbent (or gas-getting) layer 122 and a non-aluminum-based dielectric layer 124. Coat structure 130, which contacts major surface 114 of substrate 110, similarly includes a non-aluminum hydrogen-absorbent (or gas-getting) layer 132 and a non-aluminum dielectric layer 134. As used herein, the term “absorb” and its derivatives includes adsorb.

In the exemplary embodiment, non-aluminum hydrogen-absorbent layers 122 and 132 consist essentially of titanium and have a substantially uniform thickness in the range of 10-1000 nanometers, for example, 500 nanometers. Dielectric (or insulative) layers 124 and 134 consist essentially of titanium oxide and have a substantially uniform thickness in the range of 0.5-5.0 nanometers. (As used herein the term titanium oxide includes any form of oxidized titanium and thus encompasses, for example, one or more of the following: TiO, TiO₂, Ti₂O₃ and Ti₃O₅.) Notably, the combination of aluminum and titanium exhibits an increase hydrogen solubility compared to pure titanium, exhibiting for example a hydrogen solubility of 180-310 parts per million (ppm) at room temperature. Titanium oxide has a dielectric constant that ranges from 28 to 60, exceeding the 7-10 range associated with aluminum oxide.

Other embodiments use titanium-based alloys, titanium-containing compositions, or other gas-absorbent materials, such as palladium, zirconium, niobium, vanadium, and combinations of these materials, that also absorb hydrogen. Some embodiments use palladium-, zirconium-, niobium-, and vanadium-based alloys. Other embodiments also use other dielectrics, such as palladium oxide, zirconium oxide, niobium oxide, or vanadium oxide which may also have a higher dielectric constant than aluminum oxide.

An exemplary method of forming structure 100 entails providing an aluminum substrate, such as an aluminum foil of desired thickness and surface texture, and completely sputter coating one or both sides of the substrate with titanium to the desired uniform thickness. An exemplary titanium source has a purity of 99.5 percent. Some embodiments may mask off sections of the foil to prevent adherence of the titanium coat and thus define coated and non-coated regions. Still other embodiments may apply titanium to achieve a thickness gradient. Other embodiments may use other physical- or chemical-vapor deposition techniques to deposit the titanium.

Formation of the titanium oxide in the exemplary embodiment entails exposing the titanium-coated aluminum substrate to ambient air; however, other embodiments use other procedures for forming the titanium oxide. For instance, some may form the oxide under more specific oxygenated, pressurized, and temperature-controlled conditions.

### Exemplary Flat Capacitor

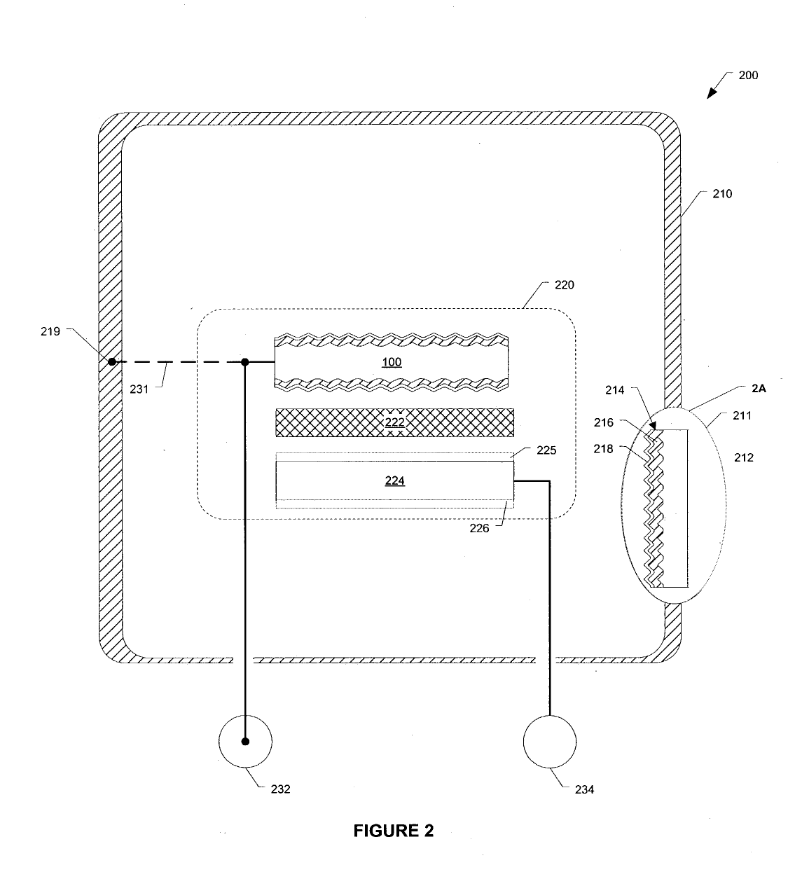

FIG. 2 shows a pictorial cross-section of an exemplary flat aluminum electrolytic capacitor 200, incorporating exemplary structure 100. Capacitor 200 includes a flat-form or pan-type case 210, a capacitor module 220, and capacitor terminals 230 and 232.

Case 210, which has a D-shape (not visible in this cross-sectional view), includes at least one wall portion 211. Wall portion 211, as shown in inset 2A, includes an aluminum substrate 212 which is affixed to a coat structure 214. In the exemplary embodiment, the interface between substrate 212 and coat structure 214 is etched; however, in other embodiments, the interface is smooth or unreached. Coat structure 216, which is similar in form and function to structure 100, includes a non-aluminum hydrogen- or gas-ion-getting layer 216 and a non-aluminum-based dielectric 218. In the exemplary embodiment, substrate 212 comprises titanium, and non-aluminum-based dielectric layer 218 comprises titanium oxide. Coat structure 216 is subject to similar material and form variations as structure 100.

Capacitor module 220, generally representative of one or more stacked capacitor modules, includes a cathodic electrode structure 100′, a separator structure 222 and an anodic electrode structure 224. Specifically, cathodic electrode structure (or cathode) 100′ has the same structural format and material composition as structure 100. Separator structure 222, which is impregnated with an electrolyte, such as an ethylene-glycol base combined with polyphosphates or ammonium pentaborate, separates cathodic electrode structure 100′ from anodic electrode structure 224. Anodic electrode structure (anode) 224 includes one or more conductive layers, although only one layer is depicted in the simplified figure. For example, some embodiments provide an anodic structure having three or more stacked conductive layers. Additionally, anodic electrode structure 224 may itself include a coat structure based on that of structure 100, as indicated by broken-line layers 225 and 226.

In the exemplary embodiment, cathodic electrode structure 100′ has a capacitance greater than that of anodic electrode structure 224. For example, the cathode capacitance is 100-1000 micro-Farads per square centimeter, and the anode capacitance is 0.8-1.4 micro-Farads per square centimeter. And, separator structure 222 comprises one or more layers of kraft paper impregnated with an electrolyte. Other embodiments, however, use other types of separators. Also, some embodiments include additional separator structures to separate capacitor module 220 from conductive elements in other capacitor modules and/or from portions of capacitor case 210. Still other embodiments include a heterogeneous set of capacitor modules, with one or more of the modules incorporating teachings of structure 100.

Coupled to electrode structures 100′ and 224 are capacitor terminals 230 and 232 Capacitor terminal 230 is coupled to cathodic electrode structure 100′, and capacitor terminal 232 is coupled to anodic electrode structure 224. In some embodiments, cathodic electrode structure 100′ is electrically coupled to case 210 at a connection point 219. FIG. 2 shows this electrical connection as a broken line 233.

In operation, capacitor 200 generally functions in a conventional manner, with the exception that the cathodic electrode structure and/or case-wall structure provide one or more performance advantages. For example, during charging and discharging of the capacitor, interaction of the electrolyte with the cathodic electrode frees hydrogen ions from the electrolyte, and some of these hydrogen ions pair up or unite to form H₂ molecules, or hydrogen gas. In contrast to conventional aluminum electrolytic capacitors that allow this hydrogen gas to accumulate and exert a mounting pressure on the capacitor case and internal capacitor components, the titanium material in the capacitor, particularly the titanium in the cathodic electrode structure, absorbs hydrogen ions and/or hydrogen gas and thus reduces or eliminates the mounting pressure. More precisely, it is presently believed that some portion of the adsorbed hydrogens atoms diffuse into the titanium coat structure as absorbed hydrogen and that some portion combine with the titanium to produce TiH₂ film, according to

2H_{ads}+Ti→TiH₂,

where the “ads” subscript denotes adsorbed atoms. (See A. M. Shams El. Din et. al, Aluminum Desalination 107, 265-276 (1996.)) Other embodiments may use other materials to absorb hydrogen or to absorb other gases and ions. Titanium itself may absorb gases other than hydrogen.

Moreover, the titanium oxide in the cathodic electrode structure has a higher dielectric constant than that of aluminum oxide and thus increases the capacitance of the cathodic electrode structure (assuming all other factors equal.). This increase in cathodic capacitance in turn reduces the voltage on the cathode because according to the relationship

C_{anode}×V_{anode}=C_{cathode}×V_{cathode}

where C_{anode} and C_{cathode} denote the respective capacitance of the anodic and cathodic structures and V_{anode} and V_{cathode} denote the respective voltages across the anodic and cathodic structures, V_{cathode} is inversely proportional to C_{cathode}. Since hydrogen ions are liberated from the electrolytes at a specific voltage, the reduced cathodic voltage can ultimately inhibit or prevent hydrogen-ion liberation in the first place, further reducing the accumulation of hydrogen gas and its distortion potential.

### Exemplary Cylindrical Capacitor

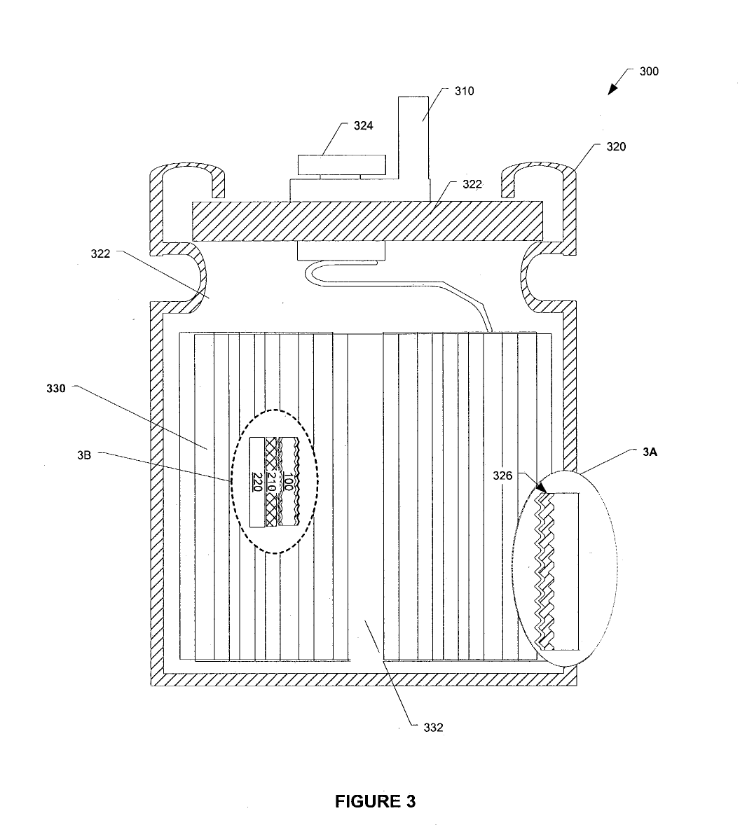

FIG. 3 shows an exemplary cylindrical aluminum electrolytic capacitor 300 which incorporates teachings of the present invention and functions in a manner similar to capacitor 200. Capacitor 300 includes terminals 310 (only one visible in this view), a case 320, and a rolled capacitor module 330.

Specifically, terminals 310 are fastened to a top or header 322 of case 320 via rivets 324 (only one visible in this view). Case 320, which consists essentially of aluminum in this exemplary embodiment, includes one or more portions that incorporate a coat structure 326 as shown in inset 3A. (Other embodiments may form the case from other metals and materials alone or in combination with each other or aluminum.) In the exemplary embodiment, coat structure 326 has a similar structural format, material composition, and functionality as that shown and/or described for coat structure 214 in FIG. 2. Rolled capacitor module 430 includes at least one elongated capacitor module, which, as inset 3B shows, has a cross-sectional structure resembling that shown and/or described for capacitor module 220 in FIG. 2. Rolled capacitor module 330 is rolled around a mandrel region 332.

### Exemplary Implantable Cardiac Rhythm Manager

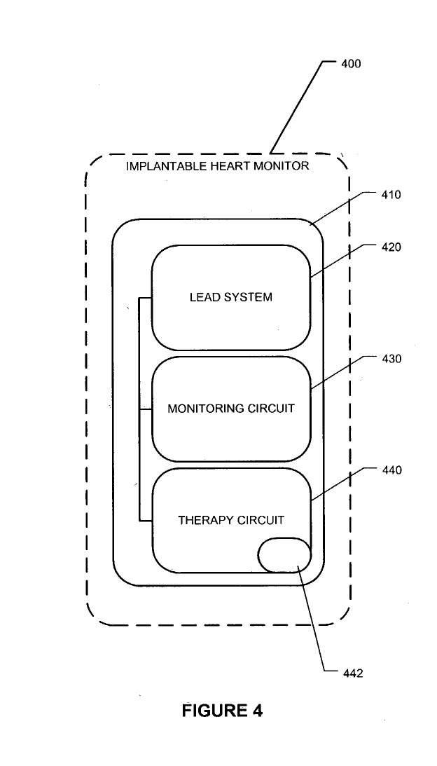

FIG. 4 shows an exemplary implantable cardiac rhythm manager 400 that includes one or more capacitors that incorporate teachings of the exemplary embodiments. Specifically, manager 400 includes a lead system 410, which after implantation electrically contact strategic portions of a patient's heart, a monitoring circuit 420 for monitoring heart activity through one or more of the leads of lead system 410, and a therapy (or pulse-generation) circuit 430 which includes one or more capacitors 432 that incorporate one or more of the teachings related to capacitor 200 or 300. Capacitors 432 are rated for an operating voltage of 390 volts and energy storage of about 14 Joules. Manager 400 operates according to well known and understood principles to generate electrical pulses and perform defibrillation, cardioversion, pacing, and/or other therapeutic or non-therapeutic functions.

### Other Exemplary Applications

In addition to aluminum electrolytic capacitors and implantable cardiac rhythm management systems or devices, the teachings of the present invention are applicable to other systems, devices, and components. For example, other types of capacitors that liberate hydrogen or other gases during operation may include the cases, anodes, and/or cathodes based on the present teachings. Also, other systems and devices that use capacitors, such as those related to photographic flash equipment, may incorporate one or more of the present teachings.

### Conclusion

In furtherance of the art, the inventors have devised not only unique structures that enhance operation of capacitors by preventing development of excessive internal pressures, but also related devices, systems, and methodologies. One exemplary capacitor includes aluminum structures coated with titanium or titanium oxide or more generally with non-aluminum-based, gas- or gas-ion-getting materials or high-dielectric-constant materials.

The embodiments described herein are intended only to illustrate and teach one or more ways of practicing or implementing the present invention, not to restrict its breadth or scope. The actual scope of the invention, which embraces all ways of practicing or implementing the teachings of the invention, is presently defined by the following claims and their equivalence.

1. An aluminum electrolytic capacitor comprising:
at least one anode; and
at least one cathode including an aluminum structure and a titanium layer contacting the aluminum structure, with the titanium layer having a thickness in the range of 10-1000 nanometers.

2. The capacitor of claim 1, wherein the thickness of the titanium layer is about 500 nanometers.

3. The capacitor of claim 1, further comprising an oxide layer contacting the titanium layer and having a thickness in the range of 0.5-5.0 nanometers.

4. The capacitor of claim 1, wherein the one anode includes three or more aluminum layers.

5. The capacitor of claim 1, wherein the capacitor is rated for a voltage at least as great as 300 volts.

6. The capacitor of claim 1, further comprising a separator structure between the one anode and the one cathode, with the separator structure impregnated with a liquid electrolyte.

7. The capacitor of claim 1, wherein the titanium layer consists essentially of titanium.

8. An aluminum electrolytic capacitor comprising:
at least one anode; and
at least one cathode including an aluminum structure and a first layer contacting at least a portion of the aluminum structure, with the first layer comprising a hydrogen-getting material and having a thickness in the range of 10-1000 nanometers.

9. The capacitor of claim 8, wherein the thickness of the first layer is about 500 nanometers.

10. The capacitor of claim 8, wherein the first layer consists essentially of palladium.

11. The capacitor of claim 8, wherein the first layer consists essentially of palladium.

12. The capacitor of claim 8, wherein the first layer consists essentially of zirconium.

13. The capacitor of claim 8, wherein the first layer consists essentially of vanadium.

14. The capacitor of claim 8, further comprising a second layer contacting the first layer, comprising an oxide, and having a thickness in the range of 0.5-5.0 nanometers.

15. The capacitor of claim 8, wherein the one anode includes three or more aluminum layers.

16. The capacitor of claim 8, wherein the capacitor is rated for a voltage at least as great as 300 volts.

17. The capacitor of claim 8, further comprising a separator structure between the one anode and the one cathode, with the separator structure impregnated with a liquid electrolyte.

18. The capacitor of claim 8, wherein the first layer consists essentially of the hydrogen-getting material.

19. An aluminum electrolytic capacitor comprising:
at least one anode; and
at least one cathode including an aluminum structure and a first layer contacting at least a portion of the aluminum structure, with the first layer comprising palladium.

20. The capacitor of claim 19, wherein the first layer has a thickness in the range of 10-1000 nanometers.

21. The capacitor of claim 19, further comprising a second layer contacting the first layer, comprising an oxide, and having a thickness in the range of 0.5-5.0 nanometers.

22. The capacitor of claim 19, wherein the one anode includes a stack of two or more aluminum layers.

23. The capacitor of claim 19, further comprising a separator structure between the one anode and the one cathode, with the separator structure impregnated with a liquid electrolyte.

24. An aluminum electrolytic capacitor comprising:
at least one anode; and
at least one cathode including an aluminum structure and a first layer contacting at least a portion of the aluminum structure, with the first layer comprising vanadium.

25. The capacitor of claim 24, wherein the first layer has a thickness in the range of 10-1000 nanometers.

26. The capacitor of claim 24, further comprising a second layer contacting the first layer and comprising an oxide.

27. The capacitor of claim 24, wherein the one anode includes a stack of two or more aluminum layers.

28. The capacitor of claim 24, further comprising a separator structure between the one anode and the one cathode, with the separator structure impregnated with a liquid electrolyte.

29. An implantable cardiac rhythm management system comprising:
one or more electrodes for coupling to a heart;
a monitoring circuit for monitoring activity of the heart through one or more of the electrodes; and
a therapy circuit for delivering electrical energy through one or more of the electrodes, wherein the therapy circuit includes at least one capacitor comprising:
at least one anode stack; and
at least one cathode including an aluminum structure and a titanium layer contacting the aluminum structure, with the titanium layer having a thickness in the range of 10-1000 nanometers.

30. The system of claim 29, further comprising an oxide layer contacting the titanium layer and having a thickness in the range of 0.5-5.0 nanometers.

31. The system of claim 29, wherein the one anode includes three or more aluminum layers.

32. The system of claim 29, further comprising a separator structure between the one anode and the one cathode, with the separator structure impregnated with a liquid electrolyte.

33. The system of claim 29, wherein the one capacitor further comprises a flat case enclosing the one anode and the one cathode.

34. An implantable cardiac rhythm management system comprising:
one or more electrodes for coupling to a heart;
a monitoring circuit for monitoring activity of the heart through one or more of the electrodes; and
a therapy circuit for delivering electrical energy through one or more of the electrodes, wherein the therapy circuit includes at least one capacitor comprising:
at least one anode; and
at least one cathode including an aluminum structure and a first layer contacting the aluminum structure, with the first layer comprising a hydrogen-getting material and having a thickness in the range of 10-1000 nanometers.

35. The system of claim 34, wherein the hydrogen-getting material includes palladium.

36. The system of claim 34, wherein the hydrogen-getting material includes niobium.

37. The system of claim 34, wherein the hydrogen-getting material includes zirconium.

38. The system of claim 34, wherein the hydrogen-getting material includes vanadium.

39. The system of claim 34, further comprising a second layer contacting the first layer and comprising an oxide.

40. The system of claim 34, wherein the one anode includes two or more aluminum layers.

41. The capacitor of claim 34, further comprising a separator structure between the one anode and the one cathode, with the separator structure impregnated with a liquid electrolyte.

42. The system of claim 34, wherein the one capacitor further comprises a flat case enclosing the one anode and the one cathode.

43. An implantable cardiac rhythm management system comprising:
one or more electrodes for coupling to a heart;
a monitoring circuit for monitoring activity of the heart through one or more of the electrodes; and
a therapy circuit for delivering electrical energy through one or more of the electrodes, wherein the therapy circuit includes at least one capacitor comprising:
at least one anode; and
at least one cathode including an aluminum structure and a first layer contacting at least a portion of the aluminum structure, with the first layer comprising palladium.

44. The system of claim 43, wherein the first layer has a thickness in the range of 10-1000 nanometers.

45. The system of claim 43, further comprising a second layer contacting the first layer and comprising an oxide.

46. The capacitor of claim 43, wherein the one anode includes a stack of two or more aluminum layers.

47. An implantable cardiac rhythm management system comprising:
one or more electrodes for coupling to a heart;
a monitoring circuit for monitoring activity of the heart through one or more of the electrodes; and
a therapy circuit for delivering electrical energy through one or more of the electrodes, wherein the therapy circuit includes at least one capacitor comprising:
at least one anode; and
at least one cathode and including an aluminum structure and a first layer contacting at least a portion of the aluminum structure, with the first layer comprising vanadium.

48. The system of claim 47, wherein the first layer has a thickness in the range of 10-1000 nanometers.

49. The system of claim 47, further comprising a second layer contacting the first layer and comprising an oxide.

50. The capacitor of claim 47, wherein the one anode includes a stack of two or more aluminum layers.

51. A method of making a cathode for an aluminum electrolytic capacitor, the method comprising:
providing an aluminum substrate; and
forming a layer comprising palladium or vanadium on the aluminum substrate.

52. The method of claim 51, wherein providing the aluminum substrate comprises etching a surface of the aluminum substrate.

53. The method of claim 51, wherein forming the layer comprises vapor deposition.
